(19) United States
(12) Patent Application Publication
(10) Pub. No.: US 2025/0160908 A1
(43) Pub. Date: May 22, 2025

(54) REMOTE CONTROL MODULE FOR ADJUSTABLE IMPLANTS

(71) Applicant: NuVasive Specialized Orthopedics, INC., San Diego, CA (US)

(72) Inventors: Shanbao Cheng, Aliso Viejo, CA (US); Jorge Lopez Camacho, Aliso Viejo, CA (US); Youngsam Bae, Aliso Viejo, CA (US); Michael Moeller, Aliso Viejo, CA (US); Gabriel Buenviaje, Aliso Viejo, CA (US); Shawn Placie, Aliso Viejo, CA (US)

(21) Appl. No.: 19/028,960

(22) Filed: Jan. 17, 2025

Related U.S. Application Data
(63) Continuation of application No. 17/405,663, filed on Aug. 18, 2021, now Pat. No. 12,213,708.
(60) Provisional application No. 63/075,640, filed on Sep. 8, 2020.

(51) Int. Cl. A61B 17/72; A61B 17/00; A61B 17/70

(52) U.S. Cl. CPC A61B 17/7216; A61B 17/70; A61B 2017/00212; A61B 2017/00221; A61B 2017/00876; A61B 2560/02; A61B 2562/0247

(57) ABSTRACT

The present disclosure broadly provides applications of communication at ultrasound frequencies to establish transcutaneous data communication between medical devices located on and/or within a body of a patient, including inter alia: features for adjustable implants including data communication, hermetic containment, and torque amplification features.

### CROSS REFERENCE TO RELATED APPLICATIONS

The present application is a continuation application of U.S. patent application Ser. No. 17/405,663, filed on Aug. 18, 2021 (published as U.S. Pat. Pub. No. 2022-0071670), which claims the benefit of U.S. Provisional Application Ser. No. 63/075,640, filed Sep. 8, 2020 (expired), each of which is incorporated by reference in its entirety as though it were fully set forth herein.

### FIELD OF DISCLOSURE

The present disclosure pertains to the field of medical devices. More specifically, the present disclosure pertains to remote control modules for adjustable implants.

### BACKGROUND

Adjustable medical implants may be used in limb lengthening and/or spinal corrective surgical procedures to treat various conditions such as limb deformities and scoliosis. Typically, these adjustable medical implants are secured to one or more bone portions and gradually adjusted over time until some desired patient outcome is achieved.

These adjustable implants and procedures do not include an accurate and non-invasive means of measurement of in vivo conditions, such as forces and pressures, present at the implant site. Particularly, after the implant is implanted and during the course of treatment.

Further, these adjustable implants and procedures do not include reliable transcutaneous communication devices or methods to achieve bidirectional communication of power/data between implants and other medical devices. This limits biofeedback and control limiting the ability of practitioners to readily understand conditions of implants on and within the body of the patient.

### SUMMARY

In one aspect, the present disclosure provides a remote control module for adjustable implants, including: a transceiver configured to send and receive data and a driver configured to adjust an adjustable implant.

In one aspect, the present disclosure provides a system for treating a deformity, including: an adjustable implant; a remote control module; and an external transceiver; wherein the external transceiver is configured to transmit treatment instructions to the remote control module, and wherein the remote control module is configured to adjust the adjustable implant in accordance with the treatment instructions.

In one aspect, the present disclosure provides a method for treating a deformity, including the steps: providing an adjustable implant within an intramedullary cavity of a bone; securing a control module to the bone; communicating adjustment instructions to the control module using an external transceiver; and adjusting the adjustable implant in accordance with the treatment instructions using the control module.

### BRIEF DESCRIPTION OF THE DRAWINGS

These and other features may be further understood by those with skill in the art upon a review of the appended drawings, wherein:

FIG. 1 shows a system in accordance with a first embodiment including a remote control module, an adjustable implant, and an external transceiver configured to communicate with one or more of the remote control module and the adjustable implant;

FIG. 2 shows a top view of a remote control module in accordance with a second embodiment; and

FIG. 3 shows a cross-sectional side view of a system in accordance with a second embodiment including a remote control module, an adjustable implant, and an external transceiver configured to communicate with one or more of the remote control module and the adjustable implant;

### DETAILED DESCRIPTION

For purposes of explanation and not limitation, details and descriptions of certain embodiments are hereinafter provided such that one having ordinary skill in the art may be enabled to practice the subject matter. These details and descriptions are representative only of certain embodiments, and a myriad of other embodiments which will not be expressly described will be readily understood by those having skill in the art upon a thorough review hereof. Accordingly, any reviewer of the instant disclosure should interpret the scope of this disclosure by the claims, and such scope shall not be limited by the embodiments described and illustrated herein.

In one aspect, the present disclosure provides a remote control module for adjustable implants. The remote control module provides local control of an adjustable implant. In some embodiments, the remote control module may provide autonomous control and automatic adjustment of the adjustable implant in accordance with instructions communicated to the remote control module by an external transceiver.

The remote control module may include one or more of: a transceiver configured to send and receive data and a driver configured to adjust an adjustable implant. The remote control module may be configured for subcutaneous placement and implantation on and within a body of a patient. For example, the control module may be configured to be secured to a bone of a patient by one or more bone screws in proximity to and/or adjacent to a location of the adjustable implant.

The driver of the remote control module may include a magnetic element configured to magnetically couple with and drive a magnetic element of an adjustable implant. In some embodiments the magnetic element of the remote control module may include an electro-magnet configured to generate a rotating magnetic field with the adjustable implant including a rotatable permanent magnet. Rotation of the rotatable permanent magnet by the driver may change a dimension of the adjustable implant.

In some embodiments, the magnetic element of the remote control module may include a first rotatable permanent magnet configured to generate a rotating magnetic field upon a rotation thereof by an actuator. The adjustable implant may also include a second rotatable permanent magnet configured to change a dimension of the adjustable implant upon a rotation thereof. The first rotatable permanent magnet of the remote control module may be magnetically coupled to the second rotatable permanent magnet of the adjustable implant, and the driver may include an actuator configured to rotate the first rotatable permanent magnet. The actuator may include for example an electric motor configured to rotate the first permanent magnet and in turn drive a rotation of the second rotatable permanent magnet thereby adjusting the adjustable implant.

The transceiver of the remote control module may include any communication device configured to communicate information from the remote control module to an external transceiver. Although embodiments of the disclosure are described herein with reference to particular forms of acoustic signal communication, e.g., ultrasonic communication, the communication may be achieved using any form of acoustic signaling including, for example: one or more of an RF signal, a vibrational signal, an infrasound signal, and an ultrasound signal. In certain cases, the acoustic signal(s) are communicated across one or more frequency bands, amplitudes, and/or sound pressure levels (SPLs) suitable for communication with the remote control module.

Bidirectional ultrasonic communication in medical implants may provide power, enhanced control, and biofeedback between the remote control module and other devices located on and within the body of the patient. In some embodiments, the transceiver of the remote control module includes an ultrasonic transducer configured to send and receive ultrasound signals. The remote control module may include a controller operably connected to one or more of the transceiver and the driver. The controller may include one or more of a microcontroller and a microcomputer, and the controller may be configured to interpret signals from the transceiver and drive the driver in accordance with adjustment instructions conveyed from the transceiver.

Information may be conveyed within the body of an organism, such as a human, in radio frequency (RF) signals, which utilize electromagnetic waves. However, RF signals experience large amounts of attenuation in aqueous tissues, and largely reflect off metallic surfaces. Ultrasound waves and ultrasound signals experience much less attenuation within the body, but may experience elevated level of reflection and attenuation in bone and across metals.

The ultrasound waves can convey information via known amplitude and phase shifting techniques. Phase-Shift Keying is a digital modulation process which conveys data by changing the phase of a constant frequency carrier wave. The modulation is accomplished by varying the sine and cosine inputs at a precise time. It is widely used for wireless LANs, RFID and BLUETOOTH (BT). Binary phase-shift keying (BPSK) or any modulation technique may be used in ultrasound communication including: On-Off Keying (OOK), Amplitude-Shift Keying (ASK) and Frequency-Shift Keying (FSK).

The frequency of ultrasound sound waves chosen to establish the bidirectional ultrasonic communication in implants may be in any frequency of ultrasound, and are generally greater than 20 kilohertz. In some embodiments, the frequency of ultrasound sound waves may be between 200 and 400 kilohertz, for example: about 300 kilohertz. Utilizing ultrasound sound waves for power and/or data transmission in medical implants may be beneficial in that (1) ultrasound sound waves have favorable propagation and less attenuation characteristics than RF through metal or solid mediums (e.g., metallic medical implants), and (2) ultrasound sound waves can transmit data transcutaneously through various aqueous tissues in animals (e.g. human skin, muscle and bone).

Once a bidirectional ultrasound communication link is established, the implant may have a power consumption of between 0.5 mW and 80 mW, 1 mW and 60 mW, and 2.0 mW and 40 mW, 10 mW, 5 mW, and any subrange thereof. The ultrasound transducer may consume about 20 mW of power when in operation. The transducer may be configured to transmit data through at least four inches of water or aqueous tissues at a rate of 5 values per second (1 kb/s) with a data reliability of 95%. Data reliability transmitted from the transducer at these power levels may be at least 95%, at least 98%, at least 99%, at least 99.9%, or 100%. “Data reliability” means reliability over 10 minutes as calculated from a bit error rate (BER).

As discussed above ultrasound signals experience elevated levels of attenuation and reflection in bone tissues. Providing the remote control module allows a system to take full advantage of the favorable transmission characteristics of ultrasound signals by providing the control module locally and outside of a bone. Also, ultrasound communication and control elements may be bulky and too large to provide in the adjustable implant itself. Providing the remote control module locally outside of the bone, allows a designer to minimize the foot print of the adjustable implant and maximize the communication capabilities of the overall system.

For example, in intramedullary nails the footprint is limited by the size of a bone of the patient. Therefore, it is beneficial to reduce the required footprint of the intramedullary nail but still necessary to provide the communications capabilities. By providing a remote control module locally, the remote control module can drive the intramedullary nail locally and communicate with an external adjustment device.

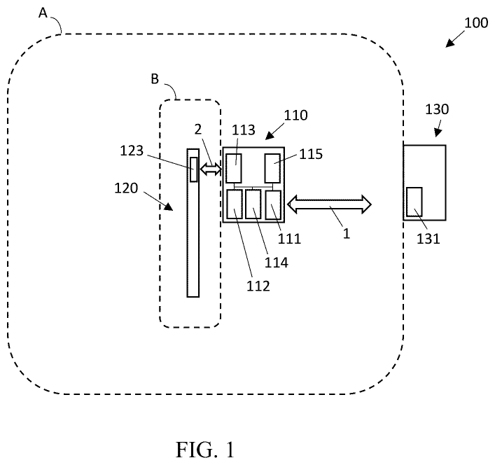

FIG. 1 shows a schematic diagram of a system 100 including an adjustable implant 120 disposed within a bone B of a patient A, a remote control module 110, and an external transceiver 130 configured to communicate with one or more of the remote control module 110 and the adjustable implant 120.

The remote control module 110 is configured to cause adjustment of the adjustable implant 120 and for transcutaneous data communication with at least the external transceiver 130. The remote control module 110 may include operatively connected circuitry including at least one transceiver 111, a sensor 112, an implant adjustment mechanism 113, a power storage device 114, a controller 115, and interconnects (not shown).

The implant adjustment mechanism 113 can include at least one rotatable permanent magnet or an electrical coil. Where the implant adjustment mechanism 113 includes at least one rotatable permanent magnet, rotation of the rotatable permanent magnet generates a magnetic field that interacts with a magnet within the adjustable implant 120 to cause adjustment of the adjustable implant 120 as will be described herein. Where the actuator 113 includes an electrical coil, the energizing of the coil creates a magnetic field that interacts with the magnet within the adjustable implant 120 to cause adjustment of the adjustable implant 120.

The controller 115 may be any type of controller including: high performance microcontrollers (MCUs), Programmable System on Chip (PSoC), Application Specific Integrated Circuit (ASIC) and any other type of controller and microcomputer. The controller 115 may be disposed on a printed circuit board which may also contain other electronic circuitry and connect other electrical components including: Analog to Digital Converter (ADC), Digital to Analog Converter (DAC), op-amps, memory, phase shifters, and any other electrical component. The controller 115 may further include a frequency synthesizer (i.e., creates carrier waves for transceivers), power amplifiers and noise filters (i.e., conditions carrier wave), power and read strain gauge (i.e., force sensor controls), and may be configured to adjust carrier waves, power, etc., such as by computer executable instructions.

In some embodiments, an energy storage device 114 may be provided. The energy storage device 114 may include a battery, a capacitor, and any other electronic charge or power storage device. The energy storage device 114 may include a rechargeable battery (e.g. Lithium ion rechargeable battery). The power storage device 114 may include a solid state battery and any battery having any known mechanism or battery chemistry. Compliance with national/regional regulatory bodies may play a part in choosing an optimal power storage device for medical implants.

The remote control module 110 may include a charging circuit operably connected to one or more of the power storage device 114 and the transceiver 111. The charging circuit may be integrated into one or more of the controller 115 and the printed circuit board. The charging circuit may include a digital switch wherein upon receiving a modulated signal at a first activation frequency the electronic switch is configured to enable charging of the power storage device 114 with electrical energy harvested by the transceiver 111. The power storage device 114 may be operably connected to the controller 115 via one or more of: wires, boards, and interconnects.

In some embodiments, known wireless charging circuits and techniques including inductive coupling and magnetic coupling may be used to wirelessly transfer power to the remote control module 110. In some embodiments one or more of data and power transfer is achieved using ultrasound signals or RF signals.

In some embodiments, the external transceiver 130 may activate the circuitry of the remote control module 110 by transmitting a modulated signal 1 to the transceiver 111. In some embodiments the modulated signal 1 is an ultrasound signal. The ultrasound signal may be received by the transceiver 111 which may include one or more ultrasonic transducer configured to convert the ultrasound signal into electrical energy. In other embodiments, the modulated signal 1 is a radiofrequency (RF) signal and the transceiver 111 may include an RF transceiver configured to convert RF energy into electrical energy. The controller 115 of the remote control module 110 may be programmed such that upon receipt of an ultrasound or RF signal (as the case may be) corresponding to a particular modulated signal, for example a particular step function of a particular temperance, the controller 115 will open/close an electrical switch and activate the remote control module 110 and place the remote control module 110 in an active and awake state. Similarly, in other embodiments a particular step function may be used to open/close the electrical switch to deactivate the remote control module 110 from the awake state to conserve power of the power storage device.

In some embodiments, the controller 115 may be programmed to time out after a certain period of time, for example if the transceiver 111 has not sent or received signals for a set period of time.

In some embodiments, the controller 115 may be programmed to turn off the power storage device 114 and to put the remote control module 110 to sleep for a certain period of time to conserve power. For example, the controller 115 may activate the remote control module 110 to transmit ultrasonic signals with 25% duty cycle. Between the pulse width, the remote control module 110 is said to be active or in awake state. The controller 115 may deactivate the remote control module 110 during the rest of the period. This state is said to be deactivated or in a sleep state.

In some embodiments the remote control module 110 may include one or more sensors 112 operably connected to the controller 115. The sensors 112 may be designed to measure one or more of: temperature, force, pressure, capacitance, resistance, and any other physical property or characteristic of the adjustable implant 120 or measure information indicative of a biological condition from surrounding anatomical structures of the patient A. The sensor 112 may include for example: a position sensor, an optical sensor, a force sensor, and any known sensor. In the instant embodiment the sensor 112 may be configured to sense force for example.

The sensor 112 may communicate a sensor reading to the controller 115, which may convert the reading to a modulated electrical signal. The modulated electrical signal may then be used to drive the transceiver 111, which then transmits a signal 1 at a frequency corresponding to the modulated electrical signal.

The controller 115 may change analogue information from the sensor 112 to digital values and may drive modulation of the transceiver 111, to transmit data using for example modulated ultrasound waves or RF signals.

The adjustable implant 120 may include any type of adjustable implant. By way of example, the adjustable implant 120 may include magnetically adjustable systems, such as the PRECICE® or MAGEC® magnetically adjustable implant systems for spinal and limb lengthening procedures sold by NuVasive, Inc. of San Diego, California. Such adjustable systems are disclosed in, for example, U.S. Pat. Nos. 9,398,925 and 9,393,117, which are incorporated by reference herein in their entireties. Further, another example of an adjustable implant 110 may include an extramedullary limb lengthening implant.

For example, the actuator of these embodiments may include a second rotatable permanent magnet 123 connected to a lead screw (not shown). Upon an axial rotation of the second rotatable permanent magnet 123 by an externally applied rotating magnetic field (e.g., a magnetic field generated by the actuator 113 of the remote control module 110), the lead screw will rotate. Rotation of the lead screw will cause an axial distraction of the adjustable implant 120, and thereby change a dimension of the adjustable implant 120.

The external transceiver 130 may include at least one ultrasonic transducer 131, including some embodiments with multiple ultrasonic transducers forming an array. A one dimensional array has multiple ultrasonic transducers disposed in a column. Each ultrasonic transducer of a one dimensional array can be assigned a position relative to their position on the array. A two dimensional array has multiple ultrasonic transducers disposed in a matrix or pattern. Each ultrasonic transducer can be assigned a location relative to two dimensions of the matrix. Each array may be a focal array with two or more of the ultrasonic transducers directed to a focal point.

Where modulated RF signals are used, the external transceiver 130 may include an antenna instead of the transducer 131 for transmitting and receiving RF signals from the transceiver 111 of the remote control module 110.

In one embodiment, a method for treating a deformity, may include the steps: providing an adjustable implant 120 within an intramedullary cavity of a bone or outside the bone (e.g., with adjustable spinal implants or with extramedullary limb lengthening implants); B; securing a control module 110 to the bone B in proximity to and/or adjacent to the adjustable implant 120; communicating adjustment and/or treatment instructions to the control module 110 using an external transceiver 130; and adjusting the adjustable implant 120 in accordance with the adjustment and/or treatment instructions using the control module 110. Such instructions can, for example, include a rate of adjustment of the adjustable implant 120, a length of adjustment of the adjustable implant 120, an amount of time of adjustment of the adjustable implant 120, a set time for the adjustable implant 120 to initiate or stop adjustment, and/or how many times adjustment of the adjustable implant 120 is initiated.

As one with skill in the art may appreciate, the remote control module 110 provides localized control and in some embodiments autonomous control of the adjustable implant 120. The external adjustment device 130, and another tertiary device including for example another implant, may communicate adjustment instructions to the remote control module 110 using one or more of an RF and an ultrasound signal 1. In some embodiments the external adjustment device 130, and another tertiary device including for example another implant, may provide remote power to one or more of the remote control module 110 and the adjustable implant 120.

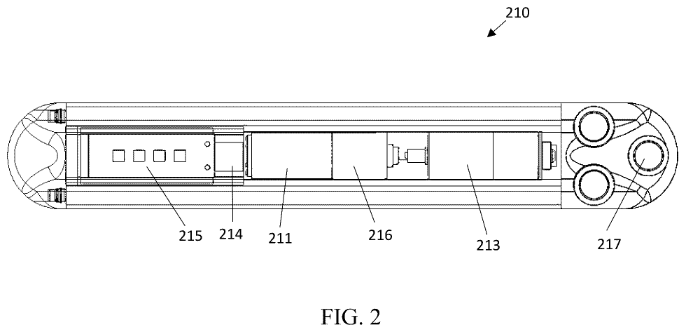

FIG. 2 shows a top view of a remote control module 210 in accordance with a second embodiment. The control module shown including a controller 215. The controller 215 is operatively connected to one or more of: an ultrasonic transceiver that is contained within or is part of controller 215, an electric motor 211, an energy storage device 214, and an optional gearbox 216. The electric motor 211 is connected to and configured to rotate a first permanent magnet 213 via the gearbox 216. Where the gearbox 216 is not included, the electric motor 211 may be directly coupled with the first permanent magnet 213. The use of the gearbox 216 may be dependent on the system torque requirements.

The remote control module 210 is configured to be mounted on a bone of a patient. The remote control module 210 includes at least one aperture 217 dimensioned to receive at least a portion of an anchor screw (i.e., bone screw) to secure the remote control module 210 to a bone in proximity to and/or adjacent to a location of the adjustable implant 220.

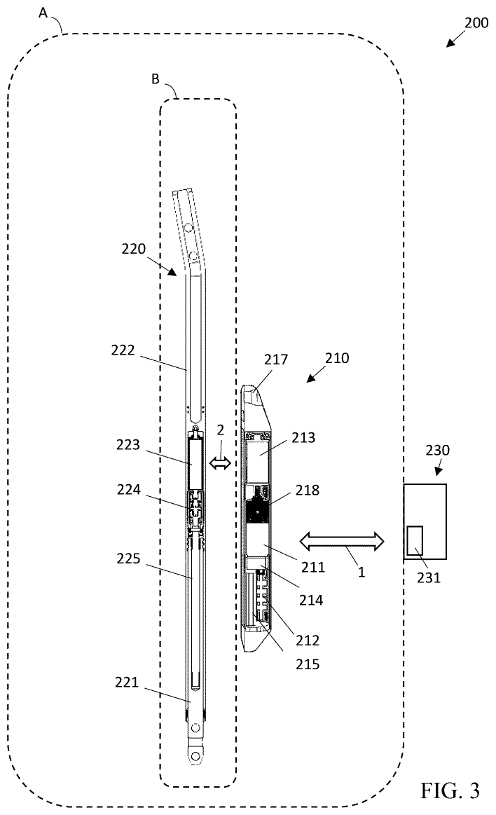

FIG. 3 shows a system 200 for treating a deformity in accordance with a second embodiment, the system 200 including a remote control module 210 and an external transceiver 230 each configured for bi-directional ultrasonic data communication. The remote control module 210 includes an actuator or electric motor 211 operatively coupled to a controller 215 and an ultrasonic transducer 212. The remote control module 210 is configured to communicate with the external transceiver 230, and the external transceiver 230 is configured to transmit adjustment instructions to the remote control module 210. The remote control module 210 is also configured to transmit data back to the external transceiver 230.

The adjustable implant 220 is shown disposed in a bone B of a patient A in a first retracted configuration with at least a portion of a rod 221 telescopically received within an outer housing 222. In this embodiment, the adjustable implant 220 is an intramedullary rod configured to treat a bone of a patient.

The remote control module 210 includes a first rotatable permanent magnet 213 configured to magnetically couple 2 with a second rotatable permanent magnet 223 of the adjustable implant 220, wherein as the first rotatable permanent magnet 213 of the remote control module 210 is rotated by the actuator or electric motor 211, the second rotatable permanent magnet 223 will be rotated thereby adjusting the adjustable implant 220.

The rod 221 of the adjustable implant 220 is configured to be telescopically received in and displaced from the outer housing 222 by a rotation of the second rotatable permanent magnet 223. In some embodiments, this adjustable implant 220 may be configured to treat another skeletal deformity, for example a distraction rod configured to be mounted to a plurality of vertebrae and configured to treat scoliosis.

As shown, the outer housing 222 and the rod 221 include apertures dimensioned to receive bone fixation devices there through, with the bone fixation devices configured to secure the implant 220 to at least one bone portion of a patient. The bone fixation devices may include bone screws, hooks, pins, rods, and any device known and used to secure an implant with respect to a patient's skeletal structure.

In this embodiment, the rod 221 includes a cavity having an at least partially threaded interior surface configured to communicate with a lead screw 225. The at least partially threaded interior surface may include a thread disposed directly on an interior surface of the rod 221. Alternatively, a nut may be positioned between the lead screw 225 and the rod 221 such that the nut is secured, e.g., via an adhesive, to the inner surface of the rod 221 and configured to engage with the threads of the lead screw 225.

The lead screw 225 is configured to rotate and displace the rod 221 relative to outer housing 222 upon a rotation of the lead screw 225 by the rotation of the second rotatable permanent magnet 223. As one with skill in the art may appreciate, depending upon the direction of rotation of the lead screw 225, the lead screw 225 may move the rod 221 into or out of the outer housing 222.

The second rotatable permanent magnet 223 is operably connected to a gear housing 224 which may include one or more planetary gear set configured to transfer rotational motion from the second rotatable permanent magnet 223 to the lead screw 225. The one or more planetary gear set may step up or step down the torque of the rotatable permanent magnet 223 depending on the intended application. In other embodiments (not shown), the second rotatable permanent magnet 223 can be directly secured to the lead screw 225 via a pin extending through an aperture within the lead screw 225 and an aperture within a magnet cup or housing surrounding the second rotatable permanent magnet 223. However, it is to be understood that the lead screw 225 and the second rotatable permanent magnet 223 can be coupled via other coupling means known in the art.

In some embodiments the lead screw 225 may include a thread pitch chosen and configured to minimize an amount of energy consumed by the electric motor 211 to move the rod 221 relative to the outer housing 222. In some embodiments, the pitch of the lead screw 225 may be chosen to optimize efficiency of the electric motor 211. Efficiency determines an amount of power consumption and indirectly defines a size of the energy storage device 214. Maximizing lead screw 225 efficiency minimizes power consumption and enabling use of a smaller energy storage device 214.

In this embodiment, a lead screw pitch of 20 threads per inch (tpi) is chosen for the small diameter lead screw 225. A lead screw pitch of 20 tpi is four times more efficient than for example a lead screw pitch of 80 tpi. The pitch of the lead screw can include any pitch known and used in the art, and may be chosen to maximize efficiency of the actuator. In some embodiments, one or more of roller screw threads and ball screw threads may be incorporated into the design to improve lead screw 225 efficiency.

In this embodiment the actuator of the remote control module 210 is an electric motor 211. The electric motor 211 is operably coupled to a controller 215. The controller 215 is also operably coupled to an ultrasonic transducer 212 and an energy storage device 214. The ultrasonic transducer 212 in this embodiment includes a hollow cylindrical ultrasonic transducer. The controller 215 may include a printed circuit board and may be configured for integration in a stacked configuration relative to the ultrasonic transducer 212 and the energy storage device 214, with the electrical connections being established through a chassis via interconnects.

One or more of the energy storage device 214 and the electric motor 211 may be hermetically sealed within the remote control module 210, and may further contained within a sealed container.

The controller 215 is configured to monitor current draw of the electric motor 211 to determine an amount of force applied by the adjustable implant 220. In this embodiment, the controller 215 may monitor the current draw of the electric motor 211 and determine an amount of force being applied to the bone B of the patient A, by monitoring the amount of current drawn by the electric motor 211 to rotate the first rotatable permanent magnet 213 in its coupled state with the second rotatable permanent magnet 223. The controller 215 may communicate the amount of force being applied to the external transceiver 230 using the ultrasonic transducer 212. And the external transceiver 230 and/or a tertiary device may use this information to calculate new distraction instructions for the adjustable implant 220 and communicate the new distraction instructions to the remote control module 220 using an ultrasonic signal 1.

Note, that some embodiments may include some of the same features as other embodiments. Accordingly, similar components share similar reference numbers with the hundreds digit changed to correspond to a specific illustrated embodiment. As one with skill in the art may appreciate, and unless noted otherwise, shared components may not be introduced in every embodiment but are identifiable by their reference numbers in similar embodiments.

Various changes, substitutions, and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present processes, machines, manufacture, composition of matter, means, methods, or steps, presently existing or later to be developed that perform the function or achieve the result as the corresponding embodiments described herein may be utilized according to the present disclosure.

What is claimed is:

1. A system for treatment of a deformity in a patient, comprising:
a control module having a transceiver; and
an adjustable implant including a driver, the adjustable implant configured to be implanted separately from the control module,
wherein the control module adjusts the adjustable implant via a magnetic field generated between the control module and the adjustable implant.

2. The system of claim 1, wherein the driver includes a first magnetic element.

3. The system of claim 2, wherein the driver includes an actuator configured to rotate the first magnetic element.

4. The system of claim 3, wherein the first magnetic element includes a rotatable permanent magnet.

5. The system of claim 3, wherein the actuator includes an electric motor.

6. The system of claim 1, wherein the transceiver includes an ultrasonic transducer configured to send and receive ultrasound signals.

7. The system of claim 1, further comprising:
a controller operably connected to one or more of the transceiver and the driver.

8. The system of claim 1, wherein the controller includes a microcontroller.

9. The system of claim 1, wherein the control module is configured for subcutaneous placement within a body of a patient.

10. The system of claim 1, wherein the control module is configured to be secured to a bone of a patient.

11. A method for treatment of a deformity in a patient, comprising the steps of:
providing an adjustable implant including a driver within a bone;
securing a control module to the bone separately from the adjustable implant;
adjusting the adjustable implant via a magnetic field generated between the control module and the adjustable implant.

12. The method of claim 11, wherein the driver includes a first magnetic element.

13. The method of claim 12, wherein the driver includes an actuator configured to rotate the first magnetic element.

14. The method of claim 13, wherein the first magnetic element includes a rotatable permanent magnet.

15. The method of claim 13, wherein the actuator includes an electric motor.

16. The method of claim 11, wherein the transceiver includes an ultrasonic transducer configured to send and receive ultrasound signals.

17. The method of claim 11, further comprising:
a controller operably connected to one or more of the transceiver and the driver.

18. The method of claim 11, wherein the controller includes a microcontroller.

19. The method of claim 11, wherein the control module is configured for subcutaneous placement within a body of a patient.

20. The method of claim 11, wherein the control module is configured to be secured to a bone of a patient.
